(19) United States
(12) Patent Application Publication
(10) Pub. No.: US 2008/0042075 A1
(43) Pub. Date: Feb. 21, 2008

(54) Thermometry apparatus probe sterilization

(75) Inventor: Timothy J. Smith, Auburn, NY (US)

Correspondence Address: Hiscock & Barclay, LLP, One Park Place, 300 South State Street, Syracuse, NY, 13202-2078, US

(73) Assignee: WELCH ALLYN, INC., Skaneateles Falls, NY (US)

(21) Appl. No.: 11/507,239

(22) Filed: Aug. 21, 2006

(51) Int. Cl. A61L 2/10

(52) U.S. Cl. 250/455.11

(57) ABSTRACT

A portable thermometry apparatus having an elongate temperature sensing probe includes a probe sterilization chamber wherein the elongate temperature sensing probe is irradiated with germicidal light to sterilize the probe between usages.

### FIELD OF THE INVENTION

This invention relates generally to electronic thermometry apparatus for measuring a patient's body temperature via a temperature sensing probe and, more particularly, to sterilization of the temperature sensing probe of such an electronic thermometry apparatus.

### BACKGROUND OF THE INVENTION

Electronic thermometry apparatus for measuring a patient's body temperature orally, rectally or axillarily via a temperature probe have been in common use in doctor's offices, clinics, hospitals and other health care facilities for many years. Typically, such medical thermometers include an axially elongate, cylindrical temperature probe having a temperature sensing element, commonly a thermocouple or thermistor, disposed within a distal tip of the probe. The temperature probe is tethered to a base housing by a flexible cord and the temperature sensing element is connected by signal conductive wires passing through the flexible cord to a processor carried in the base housing. The processor, conventionally a microprocessor, includes software for processing signals received from the temperature sensing element of the temperature probe to accurately estimate the patient's temperature. The patient's temperature is then displayed on a display, such as a LED display, carried on the base housing. Electronic thermometers of this type are disclosed in U.S. Pat. Nos. 5,632,555; 6,000,846; 6,036,361; 6,827,488 and 6,971,790, the entire contents of which are incorporated herein by reference, and are commercially available from Welch Allyn, Inc, the common assignee of the aforementioned patents and the assignee to which this application is subject to assignment.

For hygienic reasons, it has been customary practice to place a sterile disposable probe cover over the temperature probe prior to each use. The purpose of the disposable probe cover, which is discarded after a single use, is to prevent cross-contamination between patients. If the temperature probe were used without a new sterile disposable probe cover for each use, the temperature probe would have to be sterilized between each use. However, the conventional practice associated with non-electronic thermometers of immersing the thermometer in a sterilizing liquid, such as for example ethyl alcohol or ethylene oxide, for a period of time is inappropriate for use in sterilizing the temperature sensing probe of an electronic thermometry apparatus. If the sterilizing liquid where to leak into the interior of the probe, the temperature sensing element within the interior of the probe would likely be damaged, if not rendered non-functional, by contact with the liquid.

The use of ultraviolet light to sterilize certain medical instruments is known in the art. For example, U.S. Pat. No. 5,185,532 discloses a dental instrument sterilizer using ultraviolet light to sterilize dental instruments suspended in an enclosure about a centrally located ultraviolet lamp. U.S. Pat. No. 5,637,877 discloses methods and devices using diffuse ultraviolet light delivered by one or more optical fibers for sterilizing the inner surfaces of an endoscopic instrument lumen. U.S. Pat. No. 5,892,233 discloses a pocket-size device designed to simultaneously store the head of a stethoscope and to irradiate the head of the stethoscope with ultraviolet light to sterilize the head of the stethoscope. U.S. Patent Application Publication No. US2002/0162972 discloses an enclosure designed to accommodate all or portions of a single or multiple stethoscopes or other medically related re-usable items suspended therein for irradiating the suspended items with ultraviolet light. Although functional for their intended applications, none of these prior art devices are particularly adapted for or adaptable for use in connection with sterilization of the temperature sensing probe of a portable electronic thermometry apparatus.

### SUMMARY OF THE INVENTION

It is an object of an aspect of the invention to provide a portable thermometry apparatus having temperature sensing probe sterilization capability.

In is an object of an aspect of the invention to provide a portable thermometry apparatus incorporating a temperature sensing probe sterilization chamber.

In an aspect of the invention, a portable thermometry apparatus for use in determining a body temperature of a patient includes a temperature sensing probe including a longitudinally elongate member, a housing having a probe chamber adapted to receive the elongate member of the temperature sensing probe, and at least one germicidal light emitting lamp disposed in operative association with the probe chamber to irradiate the elongate member of the temperature sensing probe. In an embodiment, the at least one germicidal light emitting lamp is disposed within the interior volume of the probe well of said probe chamber. In an embodiment, the probe chamber includes a probe well defining an interior volume for retaining the elongate member of the temperature sensing probe when the temperature sensing probe is not in use and the at least one germicidal light emitting lamp may be disposed within the interior volume of the probe well of the probe chamber. In an embodiment, the probe well of the probe chamber is made from a germicidal light transmissive material, and the at least one germicidal light emitting lamp is disposed within the housing exteriorly of the probe well of the probe chamber. The at least one germicidal lamp is automatically activated upon receipt of the elongate member of the temperature sensing probe fully within the probe well of the probe chamber. The at least one germicidal lamp is operative following receipt of the elongate member of the temperature sensing probe fully within the probe well of the probe chamber to emit germicidal light for a period of time sufficient to sterilize the elongate member of the temperature sensing probe.

In an aspect of the invention, a portable thermometry apparatus for use in determining a body temperature of a patient includes a temperature sensing probe including a longitudinally elongate member, a housing having a probe chamber adapted to receive the elongate member of the temperature sensing probe, and at least one ultraviolet light emitting lamp disposed in operative association with the probe chamber to irradiate the elongate member of the temperature sensing probe with ultraviolet light. In an embodiment, the at least one ultraviolet light emitting lamp is disposed within the interior volume of the probe well of said probe chamber. In an embodiment, the probe chamber includes a probe well defining an interior volume for retaining the elongate member of the temperature sensing probe when the temperature sensing probe is not in use and the at least one ultraviolet light emitting lamp may be disposed within the interior volume of the probe well of the probe chamber. In an embodiment, the probe well of the probe chamber is made from an ultraviolet light transmissive material, and the at least one ultraviolet light emitting lamp is disposed within the housing exteriorly of the probe well of the probe chamber.

### BRIEF DESCRIPTION OF THE DRAWINGS

For a further understanding of the invention, reference will be made to the following detailed description of the invention which is to be read in connection with the accompanying drawing, where:

FIG. 1 is a perspective view of a portable thermometry apparatus equipped with a temperature sensing probe;

FIG. 2 is a side elevation view, partly sectioned, of an exemplary embodiment of the distal portion of the temperature sensing probe of the thermometry apparatus of FIG. 1;

FIG. 3 is a schematic illustration of an exemplary embodiment of a control for use in connection with the thermometry apparatus of the invention.

FIG. 4 is an elevation view, partly sectioned, illustrating an exemplary embodiment of the sterilization chamber of the thermometry apparatus of FIG. 1;

### DETAILED DESCRIPTION OF THE INVENTION

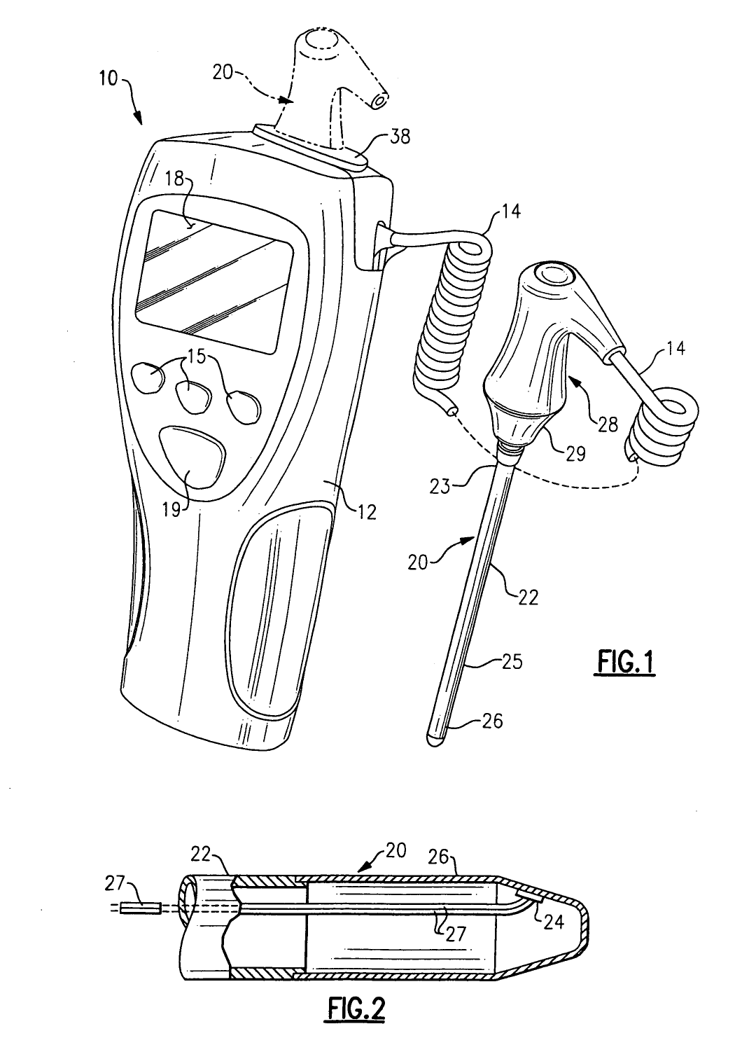

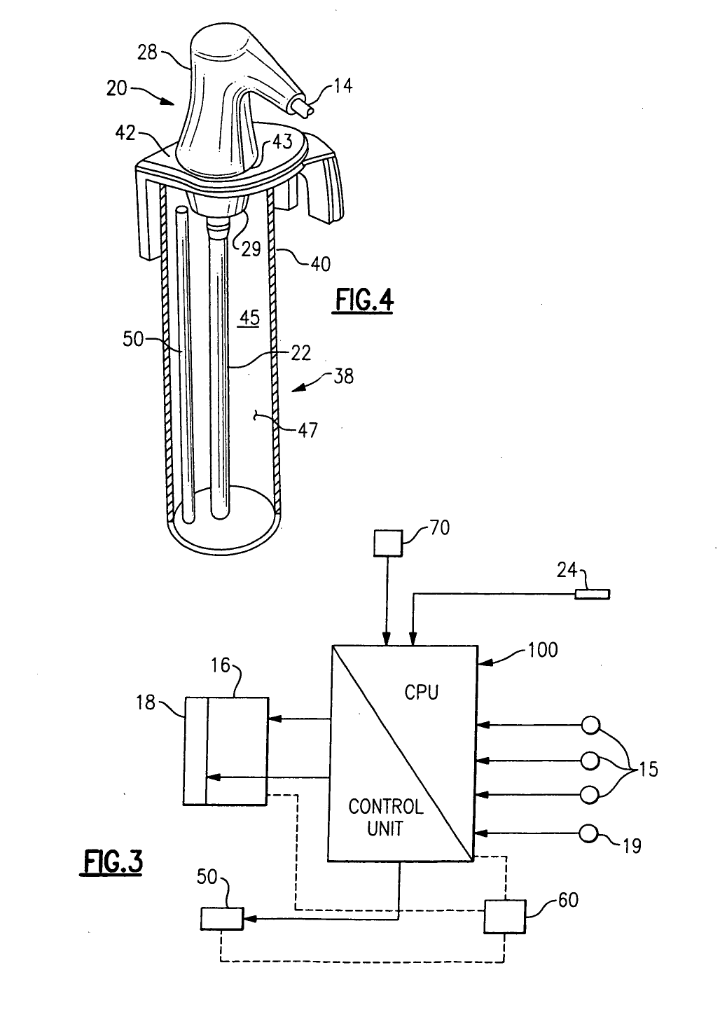

Referring now to FIGS. 1-3, there is depicted an exemplary embodiment of a portable electronic thermometry apparatus 10 for measuring a patient's temperature either orally, rectally or axillarily, and commonly referred to as a portable medical thermometer. The medical thermometer 10 includes a compact base housing 12 and a temperature sensing probe 20. The temperature probe 20 includes an axially elongate member 22 having a temperature sensing element 24, commonly a thermocouple or thermistor, disposed within a tip 26 at the distal end 25 of the elongate member 22 and having a handle 28 at the proximal end 23 of the elongate member 22. The handle 28 may include a neck portion 29 transitioning from the body of the handle to the elongated member 22. The temperature probe 20 is releasably tethered to the base housing 12 by a flexible cord 14 extending from the handle 28 of the temperature probe 20 to the base housing 12. The temperature sensing element 24 is connected by signal conductive wires 27 passing through the flexible cord 14 to a processor 100 carried in the base housing 12. The processor 100 includes software for processing signals received from the temperature sensing element 24 of the temperature probe 20 to accurately estimate the patient's temperature. A user interface operatively associated with the processor may be provided on the front of the base housing 12 that includes a display 16, such as a LED (light emitting diode) screen 18, on which the patient's temperature is displayed and one or more buttons 15 by means of which a nurse or other healthcare clinician may select the mode of temperature measurement, e.g. orally, rectally or axillarily, select between a temperature display in degrees Centigrade or degrees Fahrenheit, or set a timer alert. The display may also include a recall button 19 which may be depressed to display a prior temperature reading on the display screen 18. The thermometry apparatus 10 further includes an internal power supply 60, such as for example a set of batteries or a rechargeable power pack, housed within the base housing 12 for powering the processor, the display, and other components of the thermometry apparatus.

Referring now to FIG. 2, in particular, the elongate member 22 is a tubular, generally cylindrical member having a relatively thin wall and made from stainless steel or other thermally conductive material having relatively high strength. The tip 26 of the temperature probe 20 is also made from a stainless steel or other similar thermally conductive material and is secured to the distal end 25 of the elongate member 22, typically either by means of bonding with an epoxy or other adhesive or by laser welding. The temperature sensing element 24 is installed within the tip 26 and the signal conductive wires 27 are connected thereto and fed through the hollow interior of the shaft of the tubular elongate member 22 prior to securing the tip 26 to the distal end 25 of the tubular elongate member 22. The temperature probe 20 may also include a resistive or other heating element (not shown), advantageously disposed in the tip 26 of the probe, for pre-heating the probe tip to a desired temperature, such as for example about 93° F., in order to acclimate the probe tip prior to taking a temperature measurement, thereby reducing the overall time required to obtain an accurate temperature measurement.

The temperature probe 20 may be used to take a patient's temperature orally by placing the tip 26 of the probe 20 in a sublingual pocket beneath the tongue at the back of a patient's mouth, or axillarily by placing the tip 26 of the probe 20 in the pocket of the patient's arm pit, or rectally by insertion of the tip 26 of the elongate member 22 of the probe 20 into a patient's rectum. For a more detailed discussion of such a heating element and the other aforementioned features, as well as other features that may be included in conventional practice in a medical thermometer, reference should be made to the aforementioned U.S. Pat. Nos. 5,632,555; 6,000,846; 6,036,361; 6,827,488 and 6,971,790.

A probe chamber 38 is provided in association with the base housing 12 into which the temperature probe 20 is inserted for convenient storage when not in use. The probe chamber 38 may be mounted to the exterior of the base housing 12, for example to the rear of the base housing 12, or incorporated into the base housing 12, for example such as depicted in FIG. 2. Unlike prior art probe chambers, such as the releasable probe chamber disclosed in the aforementioned U.S. Pat. No. 6,827,488, which serve only as a storage chamber, the probe chamber 38, in accordance with the invention, is also a probe sterilization chamber. In the exemplary embodiment depicted in FIG. 4, the probe chamber 38 includes an elongate probe well 40 defining an axially elongated interior volume 45 and having a top wall 42 having an opening 43 opening to the interior volume 45. The opening 43 in the top wall is adapted to receive the neck portion 29 of the temperature sensing probe with the elongate member 22 of the temperature sensing probe 20 extending into the interior volume 45. The body of the handle 28 extends upwardly from the neck portion 29 exteriorly of the base housing 12 to provide ready access for a nurse or other user to remove the temperature sensing probe 20 from the probe chamber 38.

At least one germicidal lamp 50 is provided in operative association with the interior volume 45 for emitting light capable of providing germicidal action for killing any germs residing on the exterior surface of the elongate member 22 of the temperature sensing probe 20 suspended within the probe chamber 38. For example, the at least one germicidal lamp 50 may be, but is not limited to, an ultraviolet light emitting lamp emitting light in the ultraviolet wavelength spectrum. The at least one germicidal lamp 50 may be disposed within the interior volume 45 of the probe chamber 38 itself, such as for example extending longitudinally through the interior volume 45, as illustrated in FIG. 4, or in a separate recess (not shown) in the housing 12 adjacent to and associated with, but externally of, the probe well 40 whereby light emitted from the at least one germicidal lamp 50 disposed within the recess passes into the interior volume 45 of the probe well 40 to irradiate the elongated member 22 of the temperature sensing probe 20 suspended therein. In the latter case, the probe well 40 is made of a germicidal light transmissive material, for example a transparent or translucent material, whereby the light emitted from the germicidal lamp disposed in a recess external of the probe well 40 will pass through the probe well 40 into the interior volume 45. The at least one germicidal lamp 50 is powered by the aforementioned power supply housed within the base housing 12. Additionally, the interior surface 47 of the probe well 40 may be coated or otherwise lined with a light reflective material to ensure that the entire exterior surface of the elongated member 22 of the temperature sensing probe 40 is bathed with germicidal light irrespective of the location of the at least one germicidal lamp 50 with respect to the elongated member 22 of the temperature sensing probe 20 suspended within the interior volume 45 of the probe chamber 38. The top wall 42 of the probe well 40 and the neck portion 29 of the head 28 of the temperature sensing probe 20 received within the opening 43 in the top wall 42 prevent transmission of any ultraviolet or other germicidal light emitted from the at least one germicidal lamp from escaping the probe chamber 38.

To take a patient's temperature, the user removes the temperature probe 20 from the probe chamber 38. The thermometer 10 may be configured such that removal of the probe 20 from the probe chamber 38 automatically activates the thermometer 10, for example as described in greater detail in the aforementioned U.S. Pat. No. 6,827,488. If the thermometer 10 is not configured for automatic activation, the user will push an appropriate button (not shown) on the base housing 12 to activate the thermometer.

After verifying that the probe 20 is indeed the correct probe for the intended temperature measurement mode, i.e. orally, axillarily or rectally, and that the display 18 indicates that the thermometer 10 is in the intended temperature measurement mode, and if not manually selecting the correct temperature measurement mode, the user inserts the temperature probe 20 into the appropriate position on the patient. The user then holds the probe in position until a temperature reading appears on the display 18, typically about 10 to 15 seconds, and thereafter removes the temperature probe. The patient's temperature will be displayed on the display screen 18. When the temperature measurement process has been completed, the user reinserts the temperature sensing probe 20 into the probe chamber 38 for sterilization and storage until again needed for measuring a patient's temperature.

The thermometer 10 may be configured such that reinsertion of the probe 20 from the probe chamber 38 automatically activates the at least one germicidal lamp 50 to emit germicidal action light into the interior volume 45 of the probe chamber 38 to irradiate the elongated member 22 of the probe 20 now suspended therein. For example, a switch 70 may be provided in operative association with the probe well 40 for providing an indication of whether the temperature sensing probe 20 is stowed within the probe well 40 or is removed from the well. By monitoring the switch 70, the processor 100 determines when the temperature sensing probe is reinserted fully within the probe well 40 and activates the germicidal lamp 50 upon reinsertion of the temperature sensing probe fully within the probe well. The switch 70 may be two-position, such as for example the two-position switch disclosed in the aforementioned U.S. Pat. No. 6,827,488, that moves from a first position to a second position when the temperature sensing probe 20 is removed from the probe well 40 and returns from the second position to the first position when the temperature sensing probe 20 is reinserted into the probe well 40. The processor may be programmed to automatically deactivate the at least germicidal lamp 50 after a specified period of time from activation sufficient to sterilize the exterior surface of the elongated member 22, generally at least about 15 seconds, and usually about 15 seconds to about 30 seconds. It is within the knowledge of a skilled practitioner in the art to select the length of time that the germicidal light is active based upon the surface being sterilized and the type of germs expected to be resident on that surface prior to sterilization.

When using conventional prior art medical thermometers, a probe cover must be positioned over the elongated member of the temperature sensing probe, whether used in the oral, rectal or axillary temperature measurement modes, as a necessary hygienic protection measure to guard against cross-contamination of the temperature sensing probe from patient to patient. The sterilization of the temperature sensing probe 20 of the portable thermometry apparatus of the invention in-situ when reinserted into the probe chamber 38 between usages obviates the need to use such probe covers, not only eliminating the expense associated with such covers, but also simplifying the design of the temperature sensing probe. Prior art temperature sensing probes were customarily designed with a mechanism for retaining the probe cover thereon during usage and an additional mechanism for releasing the probe cover for disposal after usage without handling of the used probe cover by the user of the thermometer. With the in-situ sterilization feature of the portable thermometry apparatus of the invention, both of the retention mechanism and the release mechanism may be eliminated.

The present invention has been particularly shown and described with reference to the exemplary embodiments as illustrated in the drawing. It will be understood by one skilled in the art that various changes in detail may be effected therein without departing from the spirit and scope of the invention as defined by the claims.

We claim:

1. A portable thermometry apparatus for use in determining a body temperature of a patient; comprising:
a temperature sensing probe including a longitudinally elongate member;
a housing having a probe chamber adapted to receive the elongate member of said temperature sensing probe; and
at least one germicidal light emitting lamp disposed in operative association with said probe chamber to irradiate the elongate member of said temperature sensing probe.

2. A portable thermometry apparatus as recited in claim 1 wherein said probe chamber includes a probe well defining an interior volume for retaining the elongate member of said temperature sensing probe when said temperature sensing probe is not in use.

3. A portable thermometry apparatus as recited in claim 2 wherein said at least one germicidal light emitting lamp is disposed within the interior volume of the probe well of said probe chamber.

4. A portable thermometry apparatus as recited in claim 2 wherein the probe well of said probe chamber is made from a germicidal light transmissive material, and said at least one germicidal light emitting lamp is disposed within the housing exteriorly of the probe well of said probe chamber.

5. A portable thermometry apparatus as recited in claim 1 wherein said at least one germicidal light emitting lamp emits light in the ultraviolet spectrum.

6. A portable thermometry apparatus as recited in claim 1 wherein said at least one germicidal lamp is automatically activated upon receipt of the elongate member of said temperature sensing probe fully within the probe well of said probe chamber.

7. A portable thermometry apparatus as recited in claim 6 wherein said at least one germicidal lamp is operative following receipt of the elongate member of said temperature sensing probe fully within the probe well of said probe chamber to emit germicidal light for a period of time sufficient to sterilize the elongate member of said temperature sensing probe.

8. A portable thermometry apparatus as recited in claim 7 wherein said at least one germicidal lamp is operative following receipt of the elongate member of said temperature sensing probe fully within the probe well of said probe chamber to emit germicidal light for a period of time of at least about 15 seconds.

9. A portable thermometry apparatus for use in determining a body temperature of a patient; comprising:
a temperature sensing probe including a longitudinally elongate member;
a housing having a probe chamber adapted to receive the elongate member of said temperature sensing probe; and
at least one ultraviolet light emitting lamp disposed in operative association with said probe chamber to irradiate the elongate member of said temperature sensing probe with ultraviolet light.

10. A portable thermometry apparatus as recited in claim 9 wherein said probe chamber includes a probe well defining an interior volume for retaining the elongate member of said temperature sensing probe when said temperature sensing probe is not in use.

11. A portable thermometry apparatus as recited in claim 10 wherein said at least one ultraviolet light emitting lamp is disposed within the interior volume of the probe well of said probe chamber.

12. A portable thermometry apparatus as recited in claim 11 wherein the probe well of said probe chamber is made from a ultraviolet light transmissive material, and said at least one ultraviolet light emitting lamp is disposed within the housing exteriorly of the probe well of said probe chamber.
